(19) United States
(12) Patent Application Publication
(10) Pub. No.: US 2021/0389581 A1
(43) Pub. Date: Dec. 16, 2021

(54) IMAGE PICKUP APPARATUS FOR ENDOSCOPE AND ENDOSCOPE

(71) Applicant: OLYMPUS CORPORATION, Tokyo (JP)

(72) Inventor: Kazuaki KOJIMA, Suwa-shi (JP)

(73) Assignee: OLYMPUS CORPORATION, Tokyo (JP)

(21) Appl. No.: 17/412,565

(22) Filed: Aug. 26, 2021

Related U.S. Application Data
(63) Continuation of application No. PCT/JP2019/017473, filed on Apr. 24, 2019.

(51) Int. Cl. G02B 23/24; G02B 23/26; H04N 5/225; H04N 7/22

(52) U.S. Cl. CPC G02B 23/2484; G02B 23/26; A61B 1/0017; H04N 7/22; H04N 5/2251

(57) ABSTRACT

An image pickup apparatus for endoscope includes an image pickup device, an optical element, an optical fiber, a fiber tube, a plurality of metal cables, a cable tube, and a wire rod wound around the cable tube and the fiber tube to integrate the cable tube and the fiber tube. The plurality of metal cables are fixed to the cable tube deformed by the wire rod. The optical fiber is not fixed to the fiber tube by the wire rod.

### CROSS REFERENCE TO RELATED APPLICATION

This application is a continuation application of PCT/JP2019/017473 filed on Apr. 24, 2019, the entire contents of which are incorporated herein by this reference.

### BACKGROUND OF IRE INVENTION

### 1. Field of the Invention

The present invention relates to an image pickup apparatus for endoscope including an image pickup device, an optical element, an optical fiber, and a plurality of metal cables and an endoscope including an image pickup apparatus for endoscope including an image pickup device, an optical element, an optical fiber, and a plurality of metal cables.

### 2. Description of the Related Art

An endoscope includes an image pickup apparatus including an image pickup device at a distal end portion of an elongated insertion section. In recent years, use of an image pickup device having a large number of pixels in an endoscope has been considered. In the endoscope in which the image pickup device having the large number of pixels is used, an amount of signals transmitted to a signal processing apparatus from an image pickup apparatus increases. Accordingly, optical signal transmission in which optical signals are transmitted through an optical fiber is preferable instead of electric signal transmission in which electric signals are transmitted through a metal cable.

Note that, even in an endoscope that transmits an image pickup signal using an optical fiber, a metal cable is also necessary for, for example, power supply to an image pickup apparatus.

International Publication No. 2018/173323 discloses an endoscope including a photoelectric composite cable in which an optical fiber and a plurality of metal cables are integrated.

Japanese Patent Application Laid-Open Publication No. 2010-42120 discloses an endoscope in which, in order to prevent damage to a plurality of cables inserted through an insertion section, the plurality of cables are bound and housed in one tube.

### SUMMARY OF THE INVENTION

An image pickup apparatus for endoscope according to an embodiment includes: an image pickup device configured to output an image pickup signal; at least one optical element configured to convert the image pickup signal into an optical signal; at least one optical fiber configured to transmit the optical signal; a fiber tube through which the optical fiber is inserted; a plurality of metal cables configured to transmit an electric signal; a cable tube through which the plurality of metal cables are inserted; and a wire rod wound around the cable tube and the fiber tube to integrate the cable tube and the fiber tube. The plurality of metal cables are fixed to the cable tube deformed by the wire rod. The optical fiber is not fixed to the fiber tube by the wire rod.

An endoscope according to an embodiment includes an image pickup apparatus for endoscope. The image pickup apparatus for endoscope includes: an image pickup device configured to output an image pickup signal; at least one optical element configured to convert the image pickup signal into an optical signal; at least one optical fiber configured to transmit the optical signal; a fiber tube through which the optical fiber is inserted; a plurality of metal cables configured to transmit an electric signal; a cable tube through which the plurality of metal cables are inserted; and a wire rod wound around the cable tube and the fiber tube to integrate the cable tube and the fiber tube. The plurality of metal cables are fixed to the cable tube deformed by the wire rod. The optical fiber is not fixed to the fiber tube by the wire rod.

### BRIEF DESCRIPTION OF THE DRAWINGS

FIG. 1 is a configuration diagram of an endoscope system including an endoscope according to an embodiment;

FIG. 2 is a sectional view of an image pickup apparatus according to a first embodiment;

FIG. 3 is a sectional view taken along a line III-III in FIG. 2;

FIG. 4 is a sectional view taken along a line IV-IV in FIG. 2;

FIG. 5 is a sectional view of an image pickup apparatus according to a second embodiment;

FIG. 6 is a sectional view taken along a line VI-VI in FIG. 5;

FIG. 7 is a perspective view of a cable tube of the image pickup apparatus according to the second embodiment;

FIG. 8 is a sectional view of an image pickup apparatus according to a third embodiment;

FIG. 9 is a sectional view taken along a line IX-IX in FIG. 8;

FIG. 10 is a sectional view of an image pickup apparatus according to a fourth embodiment;

FIG. 11 is a sectional view taken along a line XI-XI in FIG. 10;

FIG. 12 is a perspective view of an image pickup apparatus according to a fifth embodiment;

FIG. 13 is a sectional view of a fiber cable of the image pickup apparatus according to the fifth embodiment; and

FIG. 14 is a sectional view of a cable tube of an image pickup apparatus according to a sixth embodiment.

### DETAILED DESCRIPTION OF THE PREFERRED EMBODIMENTS

### Endoscope

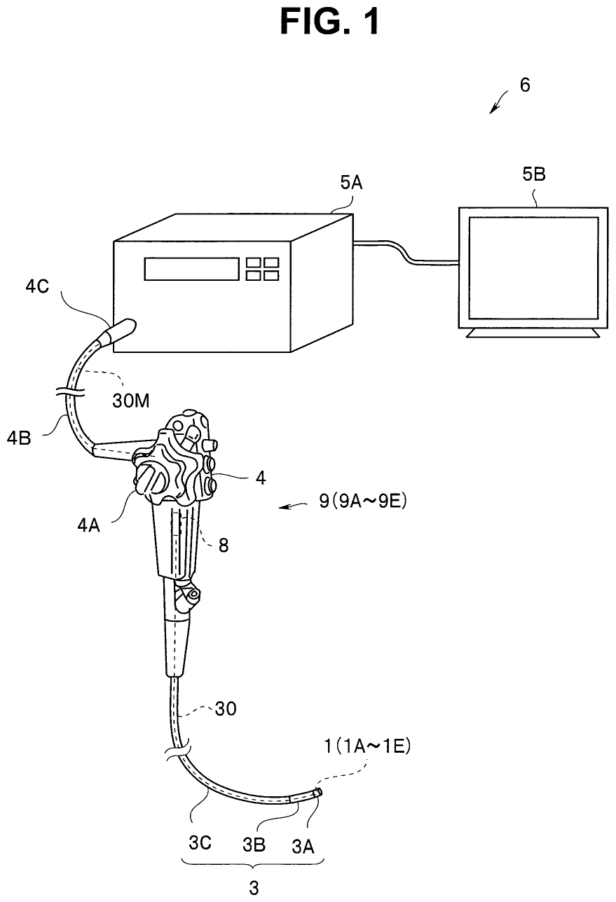

An endoscope system 6 according to an embodiment shown in FIG. 1 is configured by a processor 5A, a monitor 5B, and an endoscope 9.

The endoscope 9 includes an insertion section 3, a grasping section 4 disposed at a proximal end of the insertion section 3, a universal cord 4B extending from the grasping section 4, and a connector 4C disposed at a proximal end of the universal cord 4B. The insertion section 3 includes a distal end portion 3A, a bending portion 3B for changing a direction of the distal end portion 3A, the bending portion 3B extending from the distal end portion 3A and being bendable, and a flexible portion 3C extending from the bending portion 3B. A turning angle knob 4A, which is an operation section for a surgeon to operate the bending portion 3B, is disposed in the grasping section 4.

The universal cord 4B is connected to the processor 5A by the connector 4C. The processor 5A controls the entire endoscope system 6, performs signal processing on an image pickup signal, and outputs the image pickup signal as an image signal. The monitor 5B displays, as an endoscopic image, the image signal outputted by the processor 5A. Note that the endoscope 9 is a flexible endoscope but may be a rigid endoscope. The endoscope 9 may be either for medical use or for industrial use.

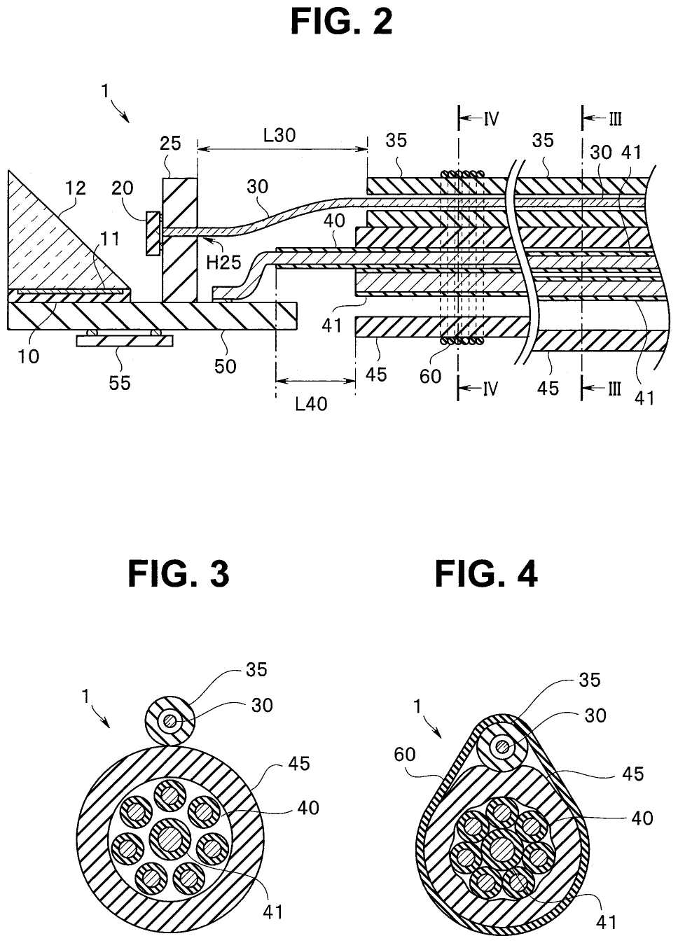

An image pickup apparatus for endoscope 1 (hereinafter referred to as an “image pickup apparatus 1” as well) is disposed at the distal end portion 3A of the endoscope 9. The image pickup apparatus 1 includes an optical element 20 (see FIG. 2 and the like) for converting an electric signal into an optical signal.

The optical signal is converted into an electric signal again by an optical module 8 disposed in the grasping section 4 by passing through an optical fiber 30, insertion of which through the insertion section 3 is allowed, and is transmitted by passing through a metal cable 30M through which the universal cord 4B is inserted. In other words, the image pickup signal is transmitted by passing through the optical fiber 30 in the small-diameter insertion section 3 and is transmitted by passing through the metal cable 30M thicker than the optical fiber 30 in the universal cord 4B not inserted into a body and having a small limitation of an outer diameter.

Note that when the optical module 8 is disposed in the connector 4C, the optical fiber 30 is inserted through the universal cord 4B.

Although not shown, a plurality of metal cables 40 and 41 (see FIG. 2 and the like) for transmitting electric power or a control signal to the image pickup apparatus 1 are also inserted through the insertion section 3 and the universal cord 4B.

As explained below, the image pickup apparatus 1 is inexpensive and has high reliability. Accordingly, the endoscope 9 is inexpensive and has high reliability.

### First Embodiment

As shown in FIG. 2, the image pickup apparatus for endoscope 1 according to the present embodiment includes an image pickup device 10, an optical element 20, an optical fiber 30, a fiber tube 35, a plurality of metal cables 40 and 41, a cable tube 45, and a wire rod 60. Note that a distal end portion of the metal cable 41 (see FIG. 3) disposed in a center in FIG. 2 is disposed in a position different from a cut surface in FIG. 2. Therefore, the distal end portion is displayed as if the distal end portion is cut halfway.

The wire rod 60 only has to be a thread that generates tension by being wound. More specifically, the wire rod 60 is a thread made of nylon, a polybenzazole fiber, a polyphenylene sulfide fiber, an aramid fiber, a silicon carbide-based fiber, a carbon fiber, a silicon fiber, a polyarylate fiber, ceramic, titanium, tungsten, polyethylene terephthalate (PET), polypropylene (PP), or the like or a blended yarn using at least two kinds of threads among the foregoing.

In the following explanation, drawings based on respective embodiments are schematic. Relations between thicknesses and widths of respective portions, ratios of the thicknesses of the respective portions, and the like are different from real ones. Portions, relations and ratios of dimensions of which are different, are included among the drawings. Illustration of a part of constituent elements and imparting of reference numerals and signs to a part of the constituent elements are omitted. A distal end direction of the insertion section 3 of the endoscope 9 is referred to as “front”.

An object image condensed by an optical system (not shown) is reflected by a prism 12 and made incident on a light receiving region 11 of the image pickup device 10. Light made incident on the image pickup device 10, which is a CCD or CMOS light receiving element, is photoelectrically converted into an image pickup signal. The image pickup signal is converted into a driving signal for driving the optical element 20 by an electronic component such as a driving IC 55.

The optical element 20 is a light emitting element that converts the image pickup signal (the driving signal) into an optical signal. The fiber 30 transmits the optical signal outputted by the optical element 20. The optical element 20 is mounted on a holding member 25. The optical fiber 30 is inserted into an insertion hole H25 of the holding member 25. in other words, in the holding member 25, the insertion hole H25 is formed in a position corresponding to the mounted optical element 20. The optical fiber 30 inserted into the insertion hole H25 is optically coupled to the optical element 20.

The plurality of metal cables 40 and 41 transmit electric power or a control signal to the image pickup device 10 and the electronic component.

The image pickup device 10, the electronic component such as the driving IC 55, and the holding member 25 are disposed on a wiring board 50. The wiring board 50 includes FPC, ceramic, glass epoxy, glass, or silicon as a base. The plurality of metal cables 40 and 41 are bonded to electrodes (not shown) of the wiring board 50 to thereby be electrically connected to the image pickup device 10 and the electronic component.

The optical fiber 30 is inserted through the fiber tube 35, For example, the optical fiber 30 is 100 μm in outer diameter and the fiber tube 35 is 200 μm in inner diameter and 400 μm in outer diameter.

The plurality of metal cables 40 and 41 are inserted through the cable tube 45. For example, seven metal cables 40 are 300 μm in outer diameter, the metal cable 41 in a center is 400 μm in outer diameter, and the cable tube 45 is 1000 μm in inner diameter and 1500 μm in outer diameter.

The wire rod 60 is wound around the cable tube 45 and the fiber tube 35 to integrate the cable tube 45 and the fiber tube 35. For example, the wire rod 60 is a thread which is 20 μm in diameter. A specific material of the thread is as explained above. An adhesive may also be disposed in a region Where the wire rod 60 is wound, a so-called thread winding region.

The cable tube 45 and the fiber tube 35, which are integrated, are easily handled.

As shown in FIG. 3, on a rear end side relative to a fixing region where the wire rod 60 is wound, there are gaps between the plurality of metal cables 40 and 41 and the cable tube 45. The plurality of metal cables 40 and 41 are not fixed to the cable tube 45. In contrast, as shown in FIG. 4, in the fixing region where the wire rod 60 is wound, the cable tube 45 made of soft resin is deformed by the wire rod 60. Accordingly, there is no gap between the plurality of metal cables 40 and 41 and the cable tube 45. The plurality of metal cables 40 and 41 are fixed to the cable tube 45. In other words, the wire rod 60 is wound in a tension state in which the cable tube 45 is deformed.

In a state in which the plurality of metal cables 40 and 41 move back and forth in the cable tube 45, it is not easy to bond the metal cables 40 and 41 to the wiring board 50. In the image pickup apparatus 1, since the metal cables 40 and 41 are fixed, bonding work is easy. A projection amount L40 of the plurality of metal cables 40 and 41 from a distal end of the cable tube 45 is set to predetermined values of the plurality of metal cables 40 and 41 considering bonding positions to the wiring board 50 before the plurality of metal cables 40 and 41 are fixed by the wire rod 60. Note that a projection amount L30 of the optical fiber 30 from a distal end of the fiber tube 35 is preferably larger than the projection amount L40 because a curvature radius increases.

The fiber tube 35 made of hard resin is not deformed even in the fixing region where the wire rod 60 is wound. In other words, the optical fiber 30 is not fixed to the fiber tube 35.

The fiber tube 35 made of the hard resin has higher rigidity and is less easily deformed than the cable tube 45 made of the soft resin. Therefore, the fiber tube 35 is not deformed even if the wire rod 60 is wound around the fiber tube 35.

Accordingly, stress is not applied to the optical fiber 30 from the wire rod 60. Note that the wire rod 60 may be wound in a state in which the optical fiber 30 is not projected from the distal end of the fiber tube 35. For example, the plurality of metal cables 40 and 41 may be bonded to the wiring board 50 after the wire rod 60 is wound. Thereafter, the optical fiber 30 may be projected from the distal end of the fiber tube 35.

The fiber tube 35 is made of PEEK resin, a Young's modulus of which is 20 GPa. In contrast, the cable tube 45 is made of silicone resin, a Young's modulus of which is 0.1 GPa. Note that when a PET thread is used as the wire rod 60, a Young's modulus of the PET thread is 2.5 GPa, The Young's moduli were measured at 25° C. according to ASTM-D638.

If rigidity of the fiber tube 35 is higher than rigidity of the cable tube 45, both of the fiber tube 35 and the cable tube 45 may be made of resin of the same kind. For example, rigidity of fluorocarbon resin or silicone resin greatly changes according to structure and an additive, Further, even if the cable tube 45 is made of not soft resin but resin having relatively high rigidity, the fiber tube 35 only has to be made of resin having higher rigidity. However, in this case, it is necessary to increase tension at a time when the wire rod 60 is wound.

Rigidify of a tube changes according to not only a kind of resin but also a thickness of the resin. Even if the fiber tube 35 is made of the same resin as the cable tube 45, by setting a thickness of the fiber tube 35 sufficiently larger than a thickness of the cable tube 45, the rigidity is also increased. Therefore, it is possible to obtain the same effects as the effects in the present embodiment.

However, when the thickness of the tube is large, it is not easy to reduce a diameter of an endoscope. Accordingly, it is preferable to use the fiber tube 35 and the cable tube 45 made of kinds of resin having different rigidities.

The image pickup apparatus 1 is inexpensive because an expensive photoelectric composite cable is not used. The image pickup apparatus 1 has a configuration in which a fiber and the like are easily repaired. In the image pickup apparatus 1, the fibber tube 35 and the cable tube 45 are integrated. Further, the metal cables 40 and 41 are fixed to the cable tube 45. Accordingly, bonding work for the metal cables 40 and 41 is easy and the image pickup apparatus 1 is easily manufactured. Since stress from the wire rod 60 is not applied to the optical fiber, the image pickup apparatus 1 has high reliability. The endoscope 9 including the image pickup apparatus 1 is inexpensive and easy to repair and has high reliability.

### Second Embodiment

Image pickup apparatuses for endoscope 1A to 1E according to embodiments explained below are similar to the image pickup apparatus 1 according to the first embodiment and have the same effects as the effects of the image pickup apparatus 1, Accordingly, components haying the same functions as the functions of the components of the image pickup apparatus 1 are denoted by the same reference numerals and signs and explanation of the components is omitted.

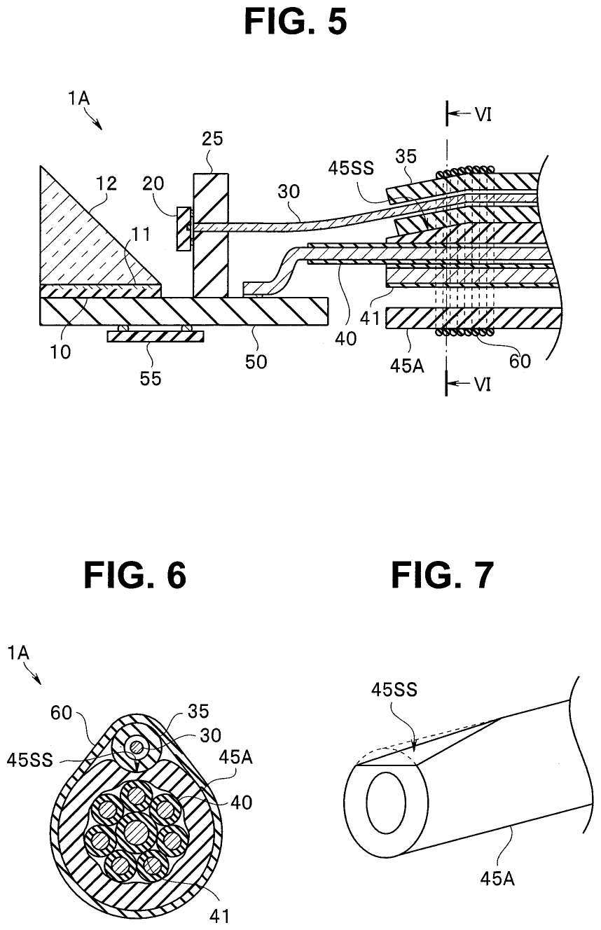

As shown in FIG. 5, FIG. 6, and FIG. 7, in the image pickup apparats for endoscope 1A according to the present embodiment, a cutout surface 45SS inclined toward a distal end center of a cable tube 45A is formed in the cable tube 45A, The fiber tube 35 is in contact with the cutout surface 45SS, whereby the optical fiber 30 is bent in a direction approaching the optical element 20.

As explained above, when an optical fiber is bent into a small curvature radius state, it is likely that leak light occurs and reliability is deteriorated.

As shown in FIG. 2, in the image pickup apparatus 1, the optical fiber 30 projects from the distal end of the fiber tube 35 and is, then, bent in the direction approaching the optical element 20. In contrast, as shown in FIG. 5, in the image pickup apparatus 1A, the optical fiber 30 is bent in the direction approaching the optical element 20 from a portion inserted into the fiber tube 35.

In the image pickup apparatus 1A, the curvature radius of the optical fiber 30 is larger than the curvature radius in the image pickup apparatus 1. Therefore, the image pickup apparatus 1A has higher reliability than the image pickup apparatus 1.

Note that it goes without saying that an image pickup apparatus in which a cutout surface inclined toward a distal end center of a fiber tube is formed in the fiber tube has the same effects as the effects of the image pickup apparatus 1A. A cutout surface may be formed in both of a cable tube and a fiber tube and the cutout surfaces in the cable tube and the fiber tube may be in contact with each other.

### Third Embodiment

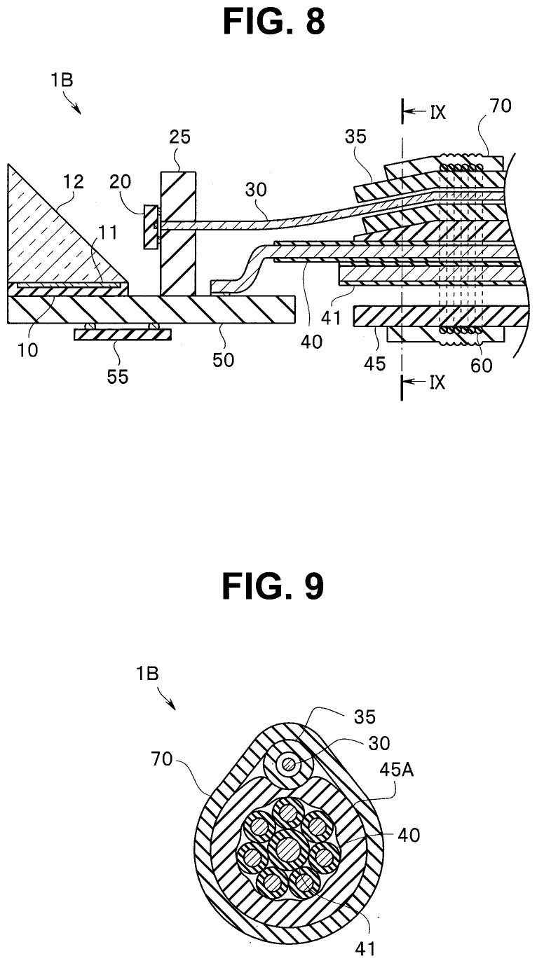

As shown in FIG. 8 and FIG. 9, in the image pickup apparatus for endoscope 1B according to the present embodiment, a fiber tube distal end region and a cable tube distal end region on a distal end side relative to a fixing region are covered by a heat shrinkable tube 70.

In the image pickup apparatus 1A, in order to bring the distal end region of the fiber tube 35 into contact with the cutout surface 45SS, it is necessary to wind the wire rod 60 around the fiber tube 35 or fix the fiber tube 35 with resin.

In a manufacturing method for the image pickup apparatus 1A, when heat is applied by a heat gun in a state in which the fiber tube 35 and the cable tube 45 are inserted through the heat shrinkable tube 70, the heat shrinkable tube 70 shrinks, whereby the distal end region of the fiber tube 35 comes into contact with the cutout surface 45SS even if tension by the wire rod 60 is absent. In other words, when the heat shrinkable tube 70 is used in this way, the wire rod 60 is not an indispensable component.

The image pickup apparatus 1B is easily manufactured because the distal end region of the fiber tube 35 is in contact with the cutout surface 45SS by the heat shrinkable tube 70. Note that the heat shrinkable tube 70 only has to cover at least a part of the cutout surface 45SS and does not always need to cover the fixing region.

### Fourth Embodiment

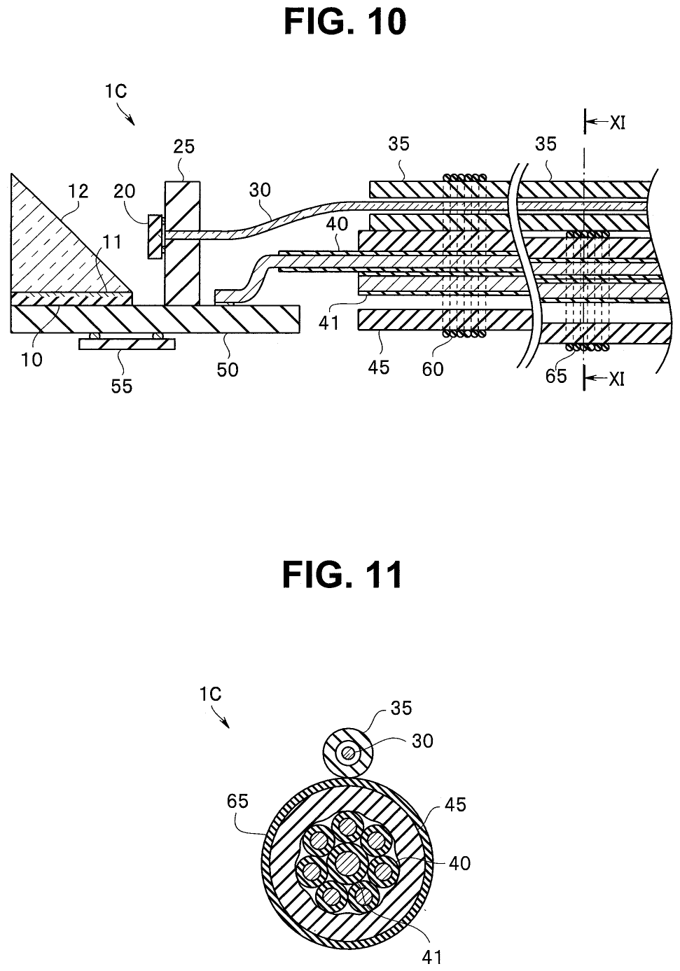

As shown in FIG. 10 and FIG. 11, the image pickup apparatus for endoscope 1C according to the present embodiment further includes a second wire rod 65 that is wound around only the cable tube 45 and fixes the plurality of metal cables 40 and 41 to the cable tube 45.

As explained above, the projection amount L40 of the plurality of metal cables 40 and 41 from the distal end of the cable tube 45 needs to be set to the predetermined values of the plurality of metal cables 40 and 41 considering the bonding positions to the electrodes of the wiring board 50. However, when the fiber tube 35 and the cable tube 45 are integrated by the wire rod 60, it is not easy to wind the wire rod 60 while maintaining the projection amount L40 of the plurality of metal cables 40 and 41.

In the image pickup apparatus 1C, by winding the second wire rod 65, the plurality of metal cables 40 and 41 are fixed in a second fixing region of the fiber tube 35 in a state of the projection amount L40 of the plurality of metal cables 40 and 41. Thereafter, the fiber tube 35 and the cable tube 45 are integrated in the fixing region (a first fixing region) by winding the wire rod 60. The first fixing region is disposed on a distal end side relative to the second fixing region.

The image pickup apparatus 1C is more easily manufactured than the image pickup apparatus 1.

### Fifth Embodiment

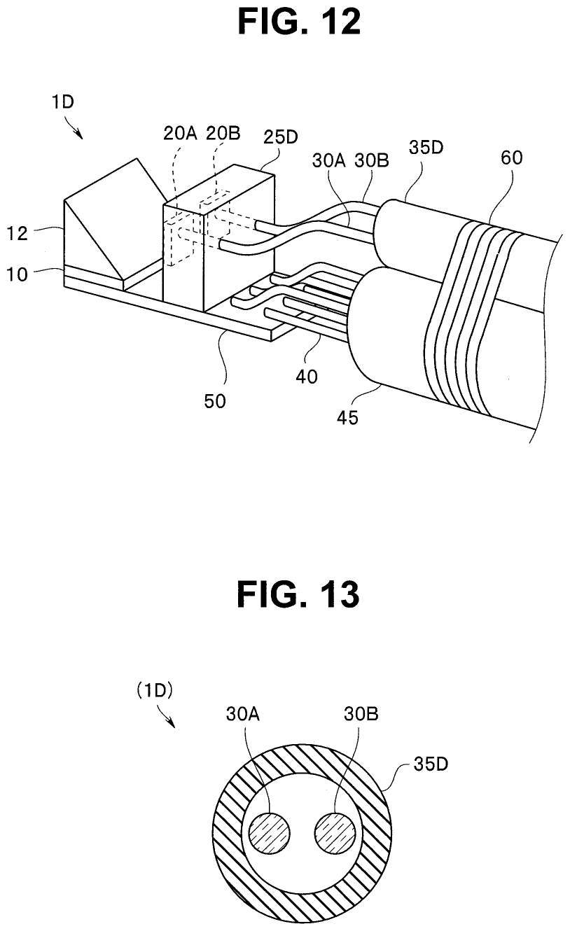

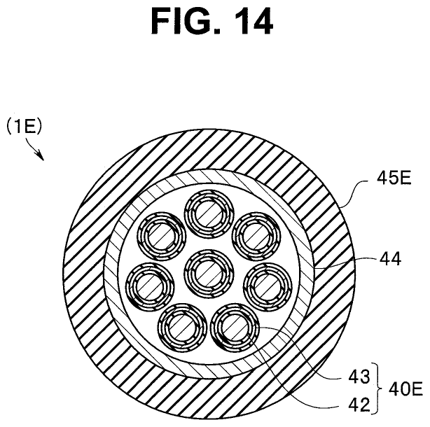

As shown in FIG. 12 and FIG. 13, the image pickup apparatus for endoscope 1D according to the present embodiment includes a plurality of optical elements 20A and 20B and a plurality of optical fibers 30A and 30B. Further, the plurality of optical fibers 30A and 30B are inserted through a fiber tube 35A.

The image pickup apparatus 1D can transmit more signals than the image pickup apparatus 1. Alternatively, the image pickup apparatus 1D is capable of performing bidirectional signal transmission.

In other words, the numbers of the optical elements and the like included in the image pickup apparatus of the present invention are not limited to one. The image pickup apparatus only has to include at least one optical element, at least one optical fiber, and a fiber tube through which the at least one optical fiber is inserted.

### Sixth Embodiment

As shown in FIG. 14, each of eight metal cables 40E of an image pickup apparatus for endoscope 1E according to the present embodiment is a shield cable including a core wire 42 and a shield layer 43. A cable tube 45E includes a second shield layer 44 that covers a plurality of metal cables 40E.

The image pickup apparatus 1E has high performance because a noise resistance characteristic of the metal cables 40E for transmitting an electric signal is excellent.

Note that it goes without saying that the configurations of the second embodiment to the fifth embodiment explained above can also be used in combination. For example, in the image pickup apparatuses for endoscope according to the embodiments, the fiber tube may be in contact with the cutout surface of the cable tube. The image pickup apparatuses may include a second wire rod wound around only the cable tube. It goes without saying that endoscopes 9A to 9E including the image pickup apparatuses 1A to 1E have the effects of the endoscope 1 and the effects of the image pickup apparatuses 1A to 1E.

The present invention is not limited to the embodiments explained above. Various changes, combinations, and applications are possible within a range not departing from the gist of the invention.

What is claimed is:

1. An image pickup apparatus for endoscope comprising:
an image pickup device configured to output an image pickup signal;
at least one optical element configured to convert the image pickup signal into an optical signal;
at least one optical fiber configured to transmit the optical signal;
a fiber tube through which the optical fiber is inserted;
a plurality of metal cables configured to transmit an electric signal;
a cable tube through which the plurality of metal cables are inserted; and
a wire rod wound around the cable tube and the fiber tube to integrate the cable tube and the fiber tube, wherein
the plurality of metal cables are fixed to the cable tube deformed by the wire rod and the optical fiber is not fixed to the fiber tube by the wire rod.

2. The image pickup apparatus for endoscope according to claim 1, wherein the fiber tube has higher rigidity than the cable tube.

3. The image pickup apparatus for endoscope according to claim 1, wherein
a cutout surface inclined toward a distal end center is formed in the cable tube. and
the fiber tube is in contact with the cutout surface, whereby an optical fiber distal end region is bent in a direction approaching the optical element.

4. The image pickup apparatus for endoscope according to claim 1, wherein a fiber tube distal end region and a cable tube distal end region on a distal end side relative to a fixing region where the wire rod is wound is covered by a heat shrinkable tube.

5. The image pickup apparatus for endoscope according to claim 1, further comprising a second wire rod wound around only the cable tube, the second wire rod fixing the plurality of metal cables to the cable tube.

6. The image pickup apparatus for endoscope according to claim 1, wherein
the image pickup apparatus includes a plurality of optical elements and a plurality of optical fibers, and
the plurality of optical fibers are inserted through the fiber tube.

7. The image pickup apparatus for endoscope according to claim 1, wherein
each of the plurality of metal cables is a shield cable including a shield layer, and
the cable tube includes a second shield layer that covers the plurality of metal cables.

8. An endoscope comprising an image pickup apparatus for endoscope, wherein
the image pickup apparatus for endoscope includes:
an image pickup device configured to output an image pickup signal;
at least one optical element configured to convert the image pickup signal into an optical signal;
at least one optical fiber configured to transmit the optical signal;
a fiber tube through which the optical fiber is inserted;
a plurality of metal cables configured to transmit an electric signal;
a cable tube through which the plurality of metal cables are inserted; and
a wire rod wound around the cable tube and the fiber tube to integrate the cable tube and the fiber tube, and
the plurality of metal cables are fixed to the cable tube deformed by the wire rod, and the optical fiber is not fixed to the fiber tube by the wire rod.
